(19) United States
(12) Patent Application Publication
(10) Pub. No.: US 2022/0280111 A1
(43) Pub. Date: Sep. 8, 2022

(54) Multi-Terminal Sensors for Thread-Based Circuitry

(71) Applicant: Trustees of Tufts College, Medford, MA (US)

(72) Inventors: Sameer Sonkusale, Lincoln, MA (US); Rachel Owyeung, Medford, MA (US); Matthew Panzer, Medford, MA (US)

(21) Appl. No.: 17/634,350

(22) PCT Filed: Aug. 11, 2020
(86) PCT No.: PCT/US2020/045763

§ 371 Date: Feb. 10, 2022

Related U.S. Application Data
(60) Provisional application No. 62/885,620, filed on Aug. 12, 2019.

(51) Int. Cl. A61B 5/00; A61B 5/01

(52) U.S. Cl. CPC A61B 5/6804; A61B 5/01; A61B 2562/0209

(57) ABSTRACT

A sensor for sensing a characteristic of a measurement region includes a first thread, a second thread, which comprises a semiconductor, a dielectric that capacitively couples the threads, and a sensing electrode that interacts with the measurement region. This interaction causes a change in an electrical characteristic of the sensing electrode, the sensing electrode is in electrical communication with one of the first and second threads.

### RELATED APPLICATIONS

This application claims the benefit of the Aug. 12, 2019 priority date of U.S. Provisional Application 62/885,620, the contents of which are herein incorporated by reference.

### FIELD OF INVENTION

The invention relates to sensors and, in particular, to flexible sensors.

### BACKGROUND

As a result of advances in miniaturization and device integration, it is now possible to have wearable sensors that provide data about the wearer more or less continuously or on demand. These sensors can be worn outside the body, in which case they are often called “smart wearable systems.” They can also be worn inside the body. In such cases, they are often called “implantable diagnostic devices.”

The devices themselves are typically integrated into a two-dimensional manifold. In some cases, the manifold is a rigid planar structure, in which case devices cannot move relative to each other. However, in many cases, the devices are integrated into a flexible two-dimensional manifold. Smart clothing, in which devices are disposed on a flexible fabric, provides an example of this.

### SUMMARY

In one aspect, the invention features an article of manufacture that includes a sensor for sensing a characteristic of a measurement region. Such a sensor includes first and second threads, with the second including a semiconductor, a dielectric that capacitively couples the first and second threads at a coupling region, and a sensing electrode that is configured to engage in an interaction with the measurement region, the interaction causing a change in an electrical characteristic of the sensing electrode and that is also in electrical communication with one of the first and second threads.

Among the embodiments of such a sensor are those in which the sensing electrode is integral with the first thread and those in which it is integral with the second thread.

In some embodiments, the sensor electrode is integral with the first thread and, in operation, an electric field modulates a current on the second thread. This electric field results from interaction of the sensor electrode with the measurement region. Among these are embodiments that also include a third thread that is capacitively coupled with the second thread. In such embodiments, an interaction of the sensor electrode with the measurement region causes a perturbation of an electric field caused by a potential applied between the first and third threads. This perturbation modulates a current on the second thread.

In still other embodiments, the sensor electrode is disposed along a current path that includes the second thread. In operation, a change in an electrical property of the sensor electrode as a result of having interacted with the measurement region modulates a current along this current path.

Also among the embodiments are those in which a loop connects to the first thread. This loop encircles the second thread in the coupling region.

Also among the embodiments of the sensor are those in which conductors connect to measurement instrumentation. These include embodiments in which first and second wires knotted around the second thread connect to measurement instrumentation for sensing current in the second thread. These also include embodiments in which third and fourth threads connected to the second thread connect to measurement instrumentation for sensing current in the second thread.

Also among the embodiments are those that include first and second conducting paths connected to the second thread. In these embodiments, the sensor electrode is in electrical communication with the second thread through first conducting path. When the sensor is operational, the second path connects to measurement instrumentation.

Embodiments further include those in which the sensor is one of many sensors that are part of a sensor fabric. In some of these embodiments, the sensors are of different kinds. The sensors are multiplexed such that an output of the sensor fabric corresponds to an output of a selected one of the sensors that comprise the sensor fabric. In others, for each sensor in the plurality, there exists a distance between a coupling region of the sensor and a sensor electrode for the sensor. These distances differ among the sensors that comprise the sensor fabric. Among these embodiments are those in which the sensors are of the same kind and those in which they are different kinds. These sensor fabrics can thus either measure the same parameter at different locations or measure different parameters at the same location.

In some embodiments, the sensor is one of a plurality of sensors in a sensor fabric. Each of these sensors also includes a first thread, a second thread that defines a semiconducting portion of a current path, a dielectric that capacitively couples the first and second threads, and a sensing electrode that is along the current path. Each of the sensing electrodes in the sensor fabric is configured to interact with a measurement region. This interaction causes a change in an electrical characteristic of the sensing electrode. Each conducting path has first and second ends, the latter being shorted together. Signals on each of the first threads cause all but one of the conducting paths to be in a non-conducting state. As a result, an output of the sensor fabric represents a measurement made by a selected one of the sensing electrodes.

Other embodiments of the article of manufacture feature a selection module and a sensor fabric including a plurality of sensors, among which is the sensor. Each of these sensors includes a first thread, a second thread that defines a semiconducting portion of a current path, a dielectric that capacitively couples the first and second threads, and a sensing electrode that is along the first thread. Each of the sensing electrodes is configured to interact with a measurement region. This interaction causes a change in an electrical characteristic of the sensing electrode. Each conducting path includes first and second ends, with the second ends being shorted together. The selection module includes transistors, each of which connects to a conducting path of a selected one of the sensors. Signals on each of the transistors in the selection module cause all but one of the conducting paths to be in a non-conducting state. As a result, an output of the sensor fabric represents a measurement made by a selected one of the sensing electrodes.

Some embodiments of the article manufacture carry out Boolean logic functions. Such embodiments include a sensor fabric including a plurality of sensors, among which is the sensor. Among these sensors are sensors that are members of a set of sensors that have been interconnected to form a logic gate. Each of the sensors that are members of the set includes first and second threads capacitively coupled by the dielectric, the second thread defining a semiconducting current path.

Some embodiments of the article manufacture carry out analog computation. Such embodiments include a sensor fabric including a plurality of sensors, among which is the sensor, Each of the sensors includes capacitively coupled first and second threads and a sensor electrode along the second thread, the second thread defining a semiconducting current path. Among the sensors in the fabric are sensors that are members of a set of sensors that have been interconnected to carry out analog computation. The analog computation includes carrying out an operation on operands. These operands are defined by electrical characteristics of sensor electrodes from different sensors in the set of sensors.

The sensors themselves can be of many types. Among the sensors are those that are configured such that the change in the electrical characteristic results from a change in temperature in sensed by the sensor electrode as a result of interaction with the measurement region, those that are configured such that the change in the electrical characteristic results from mechanical force experienced by the sensor electrode as a result of interaction with the measurement region, and those that are configured such that the change in the electrical characteristic results from interaction of the sensor electrode with a chemical species in the measurement region.

### BRIEF DESCRIPTION OF THE DRAWINGS

FIG. 1 shows a first embodiment three-terminal sensor;

FIG. 2 shows a partially-encased sensor electrode;

FIG. 3 shows the embodiment in FIG. 1 with a thread that provides a reference potential;

FIG. 4 shows a second embodiment of a three-terminal sensor;

FIG. 5 shows circuit equivalents for different locations of a source electrode as shown in FIGS. 1 and 4;

FIG. 6 shows the embodiment shown in FIG. 3 in use detecting an analyte;

FIGS. 7-9 show different coupling regions for the embodiment shown in FIG. 1;

FIGS. 10-11 show different coupling regions for the embodiment shown in FIG. 4;

FIG. 12 shows a sensor fabric implemented using the sensors of FIG. 4;

FIG. 13 shows a multiplexing arrangement for the sensor fabric of FIG. 12;

FIG. 14 shows another multiplexing arrangement for the sensor fabric of FIG. 12;

FIG. 15 shows logic gates implemented using the sensors of FIG. 4; and

FIG. 16 shows examples of analog computers implemented using the sensors of FIG. 4;

### DETAILED DESCRIPTION

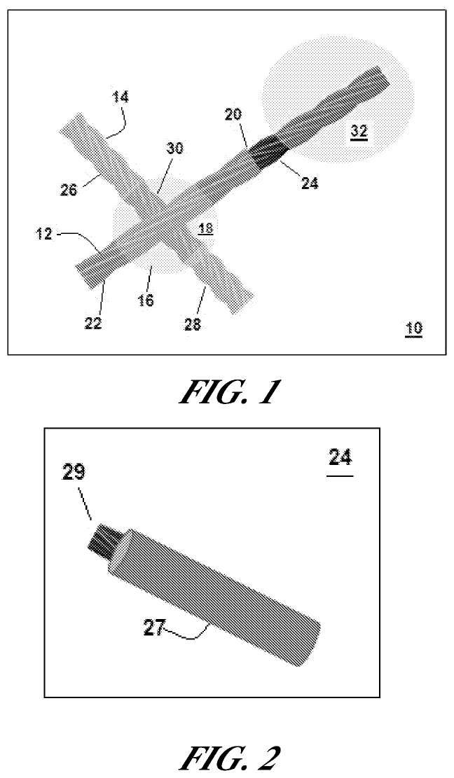

FIG. 1 shows an embodiment of a three-terminal sensor 10 formed from a first thread 12 and a second thread 14 that are capacitively coupled at a coupling region 16. A dielectric 18 separates the first and second threads 12, 14 at the coupling region 16.

As used herein, a three-terminal sensor 10 has at least three terminals. However, it is not precluded from having more than three terminals.

Each of the first and second thread 12, 14 is a semiconducting channel. Examples of a semiconducting channel include those formed by a coating or deposition of a suitable substance on a thread that acts as a substrate. Examples of suitable substances include silicon, carbon nanotubes, graphene, MoS2, WeS2, a transition-metal oxides, and a chalcogenides. Other suitable substances are organic semiconductors, among which are P3HT, PEDOT:PSS, and Pentacene. The resultant semiconducting channel is either n-type, p-type, or ambipolar.

The first thread 12 has first and second sections 20, 22 that are integral with each other. The first section 20 forms a sensing electrode 24. As such, the sensing electrode 24 and the first section 20 share the same underlying substrate. This sensing electrode 20 is one that interacts with species in solution in such a way as to modulate an electrical characteristic thereof.

In the illustrated embodiment, the sensing electrode 24 occupies only a portion of the first thread 12. However, in some embodiments, the sensing electrode 24 is intertwined or completely merged with the first thread 12.

The sensing electrode 24 is typically formed by functionalizing the second section 22. As an example, the second section 22 can be functionalized by an antibody that binds to a particular protein. Once bound to the protein, the charge distribution in the complex formed by the combination of the protein and antibody will change relative to that of the antibody in its unbound state. This change in charge distribution manifests itself as a change in the electrical characteristics of the second section 22.

In general, the sensing electrode 24 is an electrode that is deposited on an underlying substrate, that interacts with the target analyte, and that, as a result of that interaction, manifests a disturbance in its electrical properties, including potential, current, charge, and/or impedance.

In some embodiments, the sensing electrode 24 is bare metal. In others, it is carbon. Still others feature a conductive polymer electrode that senses a biopotential. Examples of suitable biopotentials that such a sensing electrode 24 can sense include action potentials from neurons. As a result, such a sensing electrode 24 is particularly useful for electrocardiograms, electroencephalograms, electromyograms, and electrooculograms.

The change in electrical characteristic can be manifested in a variety of ways. For example, in some embodiments, an accumulation of charge occurs. In others, the impedance of the second section 22 changes. As used herein, “impedance” is a complex-valued quantity whose real part is a resistance and whose imaginary part, depending on its sign, is either a capacitance or an inductance.

The sensing electrode 24 can be tuned to sense physical quantities such as strain or temperature. Among these embodiments are those in which strain or temperature modulate resistivity of a material from which the sensing electrode 24 is made.

Other embodiments of a sensing electrode 24 are tuned to detect particular gases, liquids, or volatile chemicals. Other embodiments of a sensing electrode 24 are tuned to measure quantities of analytes. Examples of analytes that can be sensed by a suitably modified sensing electrode 24 include metabolites, cytokines, including interleukin-6, nucleic acids, including both deoxyribonucleic acids and ribonucleic acids, exosomes, and neuropeptides.

In many cases, the sensing function is achieved by functionalizing with antibodies, aptamers, or complementary deoxyribonucleic acids and ribonucleic acids that are prone to interact with a particular target analyte so as to change some electrical property, such as a transfer resistance, a capacitance, or a change in current.

In some embodiments, the sensing electrode 24 is coated with polyalanine for use as a pH sensor. In others, the sensing electrode 24 is coated with an ionophore matrix to provide the sensing electrode 24 with sensitivity to only a particular ion. Examples of such ionophore matrices include those based on sodium ion and those based on ammonium ion.

The second thread 14 includes a first section 26, a second section 28, and a coupling section 30 between the first and second sections 26, 28. The first and second section 26, 28 are coated with a material that permits them to be used as measurement terminals for sensing the change in the electrical characteristic of the sensing electrode 24.

The first and second threads 12, 14 thus combine to form a three-terminal device with one terminal on the first thread 12 and two terminals on the second thread 14. The sensing electrode 24 forms the terminal on the first thread and the first and second sections 26, 28 form the two terminals on the second thread 14.

In operation, the embodiment shown in FIG. 1 operates in a manner analogous to a field-effect transistor in which the first thread 12 connects to a gate at the coupling region 16 and the second thread 14 forms a source-to-drain path. a voltage is applied between the two terminals on the second thread 14. This causes a thread current to flow through the second thread 14.

Meanwhile, a sensing electrode 24 that has been tuned to interact with a particular analyte is placed in a solution 32 having an unknown concentration of that analyte. Interaction between the analyte and the sensing electrode 24 perturbs the electrical properties of the sensing electrode 24.

In the embodiment shown in FIG. 1, the entire sensing electrode 24 is exposed. This results in a measurement over an extended region. However, in other embodiments, it may be useful to place a dielectric coating 27 over the bulk of the sensing electrode 24, leaving just a tip 29 exposed, as shown in FIG. 2.

The second section 28 communicates the perturbation to the coupling section 30. As a result, the perturbation is communicated to the first thread 12. This, in turn, disturbs the thread current that flows through the first thread 12. By monitoring this thread current, it is possible to make inferences concerning the extent to which the analyte is present in solution. Moreover, since the thread current's magnitude can be controlled by the voltage between the ends of the second thread, it is possible to amplify the effect of the perturbation, thus improving signal-to-noise ratio.

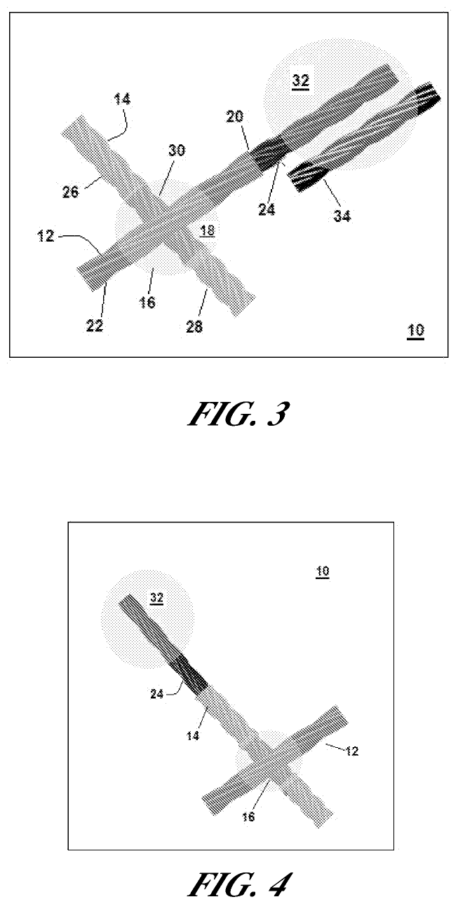

An alternative embodiment, shown in FIG. 3, features a third thread 34. This is useful if manifestation of the interaction between the sensing electrode 24 and the analyte requires that a potential difference be present. In this case, it is possible to apply a potential difference between the third thread 34 and the sensing electrode 24. Preferably, the potential at the third thread 34 also serves as a reference potential for the coupling region 16. Aside from this detail, operation of the embodiment in FIG. 2 is analogous to that described in connection with FIG. 1.

A third embodiment, shown in FIG. 4, is similar to the first embodiment but with the sensing electrode 24 having been moved to the second thread 14. In this embodiment, it is to useful to connect a time-varying voltage to the first thread 12 and to then measure the resulting thread current. The relationship between the applied time-varying voltage and the resulting time-varying thread current is indicative of any change in the imaginary part of the sensing electrode's impedance that results from having interacted with the analyte in the solution 32.

The third embodiment is analogous in structure to a field-effect transistor in which the measurement electrode 24 has been connected to a terminal other than the gate, i.e., top either the drain or source.

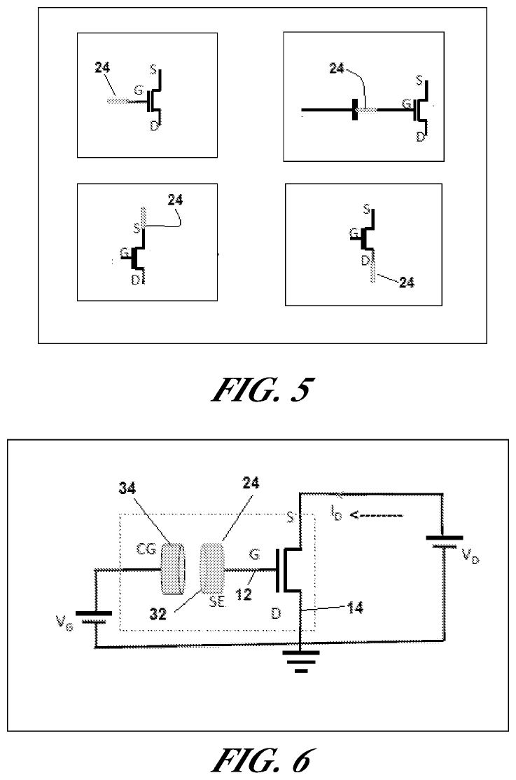

FIG. 5 show first, second, third, and fourth equivalent circuits 36, 38, 40, 42 that model the various embodiments of the three-terminal sensor identified thus far. In particular, the first circuit 36 models the embodiment shown in FIG. 1, the second circuit 38, models the embodiment shown in FIG. 3, and the third and fourth circuits 40, 42 both model the embodiment shown in FIG. 4.

FIG. 6 shows an example of using the sensing electrode 24 shown in FIG. 3 to detect an analyte. As an example, the sensing electrode 24 is wrapped in a gauze soaked in the relevant solution 32.

In this embodiment, a first voltage V_{G} is applied to the third thread 34 and a second voltage V_{D}, which is referenced to the same potential, is applied to the source terminal S associated with the second thread 14. This causes a current I_{D} from source S to drain D that can be measured. The presence or absence of analytes thus causes a change in the electric field at the gate G, which serves to modulate the measured current I_{D}.

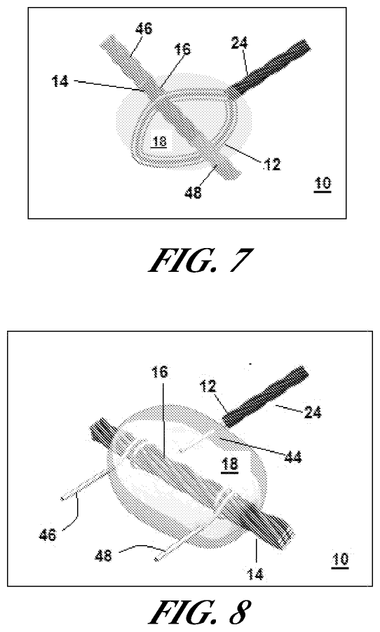

FIG. 7-9 show variations in the coupling region 16 for the embodiment shown in FIG. 1, in which the sensor electrode 24 is on the first thread 12. The coupling region 16 takes a variety of forms in addition to that shown in FIG. 1.

The coupling region 16 in FIG. 7 shows an embodiment in which the first thread 12 extends all the way around the second thread 14 at the coupling region 16. This results in capacitive coupling between the first and second threads 12, 14. In this embodiment, third and fourth conducting threads 46, 46 coupled to the second thread 14 in the coupling region 16 provide a way to connect to measurement instrumentation.

The coupling region 16 in FIG. 8 shows electrical connections made by a first wire 44 coupled to the first thread 12 and second and third wires 46, 48 that have been knotted around the second thread 14. The second and third wires 46, 48 provide a connection to measurement instrumentation.

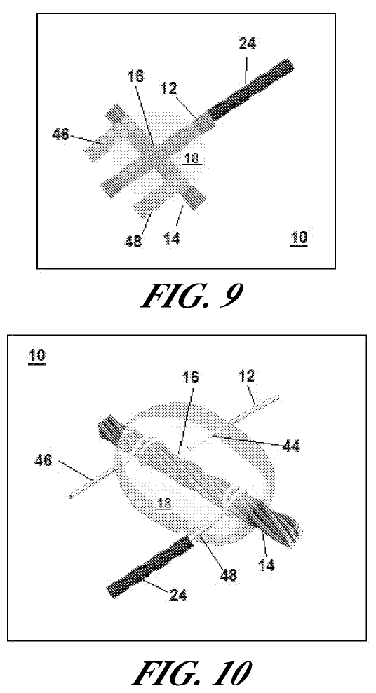

FIG. 9 features a first thread 12 that traverses the coupling region 15 and third and fourth conducting threads 46, 48 that couple to the second thread 12 in the coupling region 16. The third and fourth conducting threads 46, 48 provide a connection to measurement instrumentation.

The sensors 10 shown in FIGS. 7-9 can all be converted into the embodiment shown in FIG. 3 by addition of a third thread 34 as shown in FIG. 3.

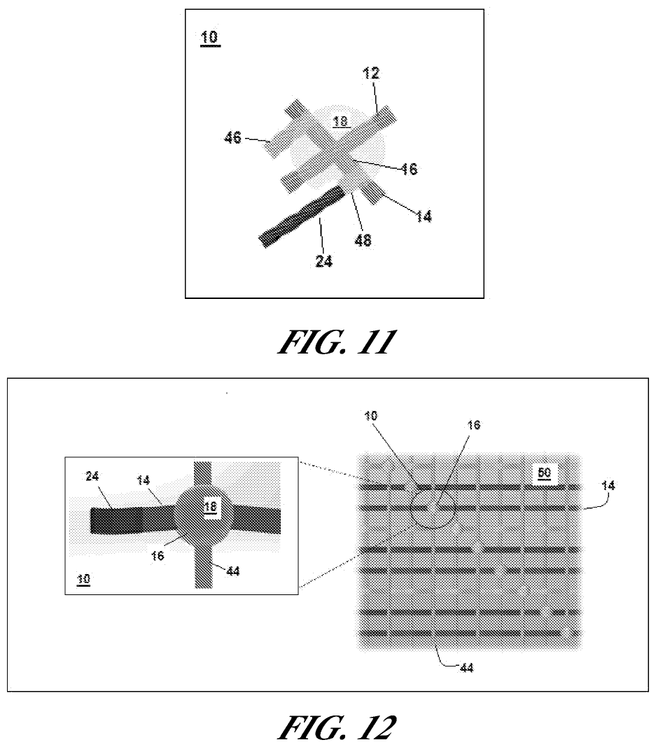

FIG. 10-11 show variations in the coupling region 16 for the sensor 10 shown in FIG. 3, in which the sensor electrode 24 is on the second thread 14. The coupling region 16 takes a variety of forms in addition to that shown in FIG. 3.

The coupling region 16 in FIG. 10 shows electrical connections made by a conducting first wire 44 that is ultimately coupled to an AC source. Conducting second and third wires 46, 48 have been knotted around the second thread 14. In this embodiment, the sensing electrode 24 capacitively couples to the second thread 14 via the third wire 48. The second wire 46 provides a connection to measurement instrumentation.

In some cases, it is useful to combine sensors 10 to form a sensor fabric 50 as shown in FIG. 11.

The sensor fabric 50 of FIG. 11 features sensors 10 of the type shown in FIG. 4 in which the sensor electrode 24 is coupled to the second thread 14. The sensor fabric 50 thus has plural first wires 44 for connection to corresponding voltage sources.

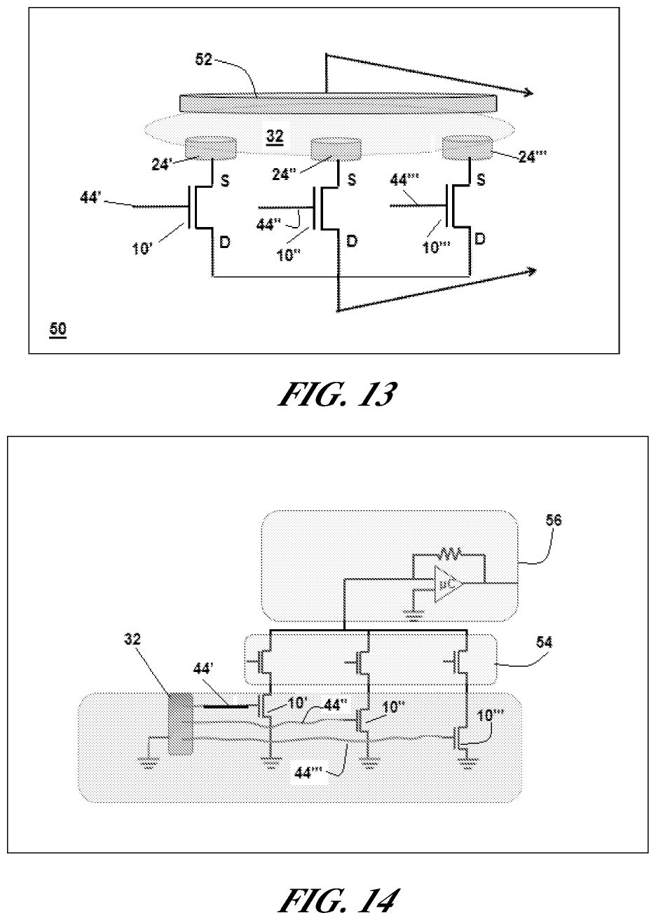

A sensor fabric 50 as shown in FIG. 11 creates the possibility of multiplexing by using the different first wires 44 to drive all but one of the sensors into a non-conducting state. A suitable interconnection for doing so can be seen in FIG. 13.

FIG. 13 shows first, second, and third sensors 10′, 10″, 10′″ with corresponding first, second, and third first-wires 44′, 44″, 44′″ and corresponding first, second, and third sensor electrodes 24′, 24″, 24′″ measuring different analytes present in the same solution 32. A reference electrode 52 applies a reference potential to the solution 32. The drains of the first, second, and third sensors 10′, 10″, 10′″ have all been shorted together. Accordingly, by applying suitable voltages to the first, second, and third first-wires 44′, 44″, 44′″, it is possible to cause only one of the first, second, and third sensors 10′, 10″, 10′″ to be in a sensing state. This makes it possible to read from only one of the first, second, and third sensor electrodes 24′, 24″, 24′″.

FIG. 14 shows an embodiment that uses first, second, and third sensors 10′, 10″, 10′″ to make measurements of different analytes in the same solution 32. Each sensor connects to a corresponding transistor in a selection module 54, the output of which comes from whichever of the first, second, and third sensors 10′, 10″, 10′″ has been selected by appropriate driving of the transistors in the selection module 54. This output is provided to an amplifier 56 that ultimately provides an amplified measurement to external circuitry. As a result, it is possible to use a single instrument to read from multiple sensors 10′, 10″, 10′″.

The first, second, and third sensors 10′, 10″, 10′″ have been described as measuring different analytes at the same general location. The result is a sensor fabric 50 that has species diversity. By having different kinds of sensor electrodes 24, it becomes possible to take measurements of many different species in the same general area and to pick out whichever measurement is of current interest at any instant by using the multiplexing architecture introduced by FIG. 13.

However, other embodiments of the sensor fabric 50 achieve spatial diversity by having the measurement electrodes 24′, 24″, 24′″ be of the same type but positioned with their sensing portions at different locations.

But there is no reason they cannot instead all be measuring the same thing but at different locations. In such cases, the sensors 24′, 24″, 24′″ are implanted at different locations. This can be achieved by having the sensors sensor 24′, 24″, 24′″ integrated into threads of different lengths. To further enhance spatial resolution, the configuration shown in FIG. 2 is particularly useful.

A sensor fabric 50 that implements a sensor array with spatial diversity is particularly useful for analysis of brain activity. In such cases, multiple sensor electrodes 24′, 24″, 24′″ for sensing electrical activity in the brain can be disposed at various locations.

As the number of multiplexed sensors 10′, 10″, 10′″ in a fabric increases, a new problem emerges. The process of turning particular sensors off and leaving only one turned on requires control electronics. Increasing the number of sensors 10′, 10″, 10′″ thus increases the number of connections that must be made to implement sensor multiplexing.

To ameliorate this difficulty, it is useful to arrange the sensors 10′, 10″, 10′″ in a binary tree. This provides the ability to address an individual sensor 10′, 10″, 10′″ using significantly fewer connections. In general, the number of connections required to address a particular sensor 10′, 10″, 10′″ increases with the number of levels in the resulting binary tree rather than with the number of sensors 10′, 10″, 10′″. More precisely, the number of connections required to address a particular sensor 10′, 10″, 10′″ out of N such sensors falls from N connections to log₂(N) connections.

For convenience, the actual switching process required to select a particular sensor 10′, 10″, 10′″ can be implemented using special logic sensors that sense a logic state. Such sensors have first and second threads 12, 14 as shown in FIG. 4 and FIG. 10 but do not require a sensor element 24. Without the sensor element 24, the logic sensor simulates the behavior of a field-effect transistor, albeit without the same speed of response. As such, it can be used as a switch. In such cases, the first wire 44 (best seen in FIG. 10) or first thread 12 (see FIG. 9) sense a desired logic state. Accordingly, a suitable potential applied to the first wire 44 or first thread 12 makes it possible to selectively turn that logic sensor on or off. This provides a convenient way to implement the selection module 54 shown in FIG. 14 without sacrificing

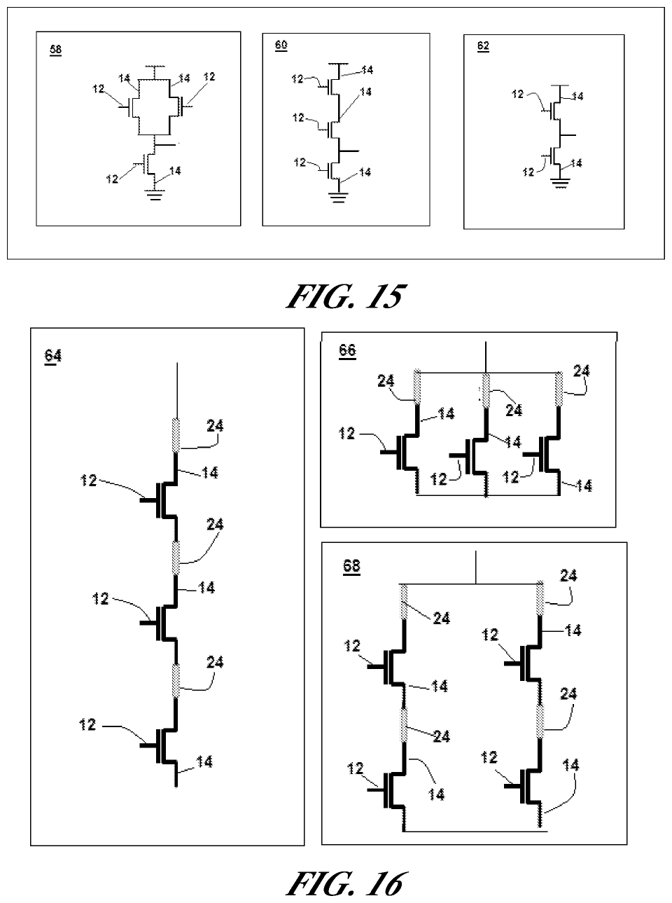

Since the logic sensors effectively simulate the operation of field-effect transistors, they can also inter-connected to each other to implement arbitrary Boolean logic expressions. This makes it possible to implement simple logic gates within the sensor fabric 50 itself. FIG. 15 shows, as examples of logic gates, a NAND gate 58, a NOR gate 60, and a NOT gate 62. As a result, it is possible to implement certain rudimentary digital computations within the sensor fabric 50 prior to sending signals to external measurement circuitry.

It is also possible to interconnect sensors 10 to implement analog computers. FIG. 15 shows first, second, and third analog computers 64, 66, 68.

The first analog computer 64 comprises sensors 13 connected with their respective second threads 14 in series. This provides a way to measure the sum of the impedances of all the sensor elements 24 disposed along the resulting conducting path.

The second analog computer 66 comprises sensors 13 connected with their respective second threads 14 in parallel. By applying suitable control voltages to the respective first threads 12, it is possible to compute the parallel impedance of any combination of two or more sensor elements 24.

The third analog computer 68 features a combination of the first and second analog computers 64. 66.

The threads 12, 14, 34 referred to herein include anything that can be stitched, sewn, knitted, or patterned. Threads can be made of linen, cotton, silk, nylon, rubber, wool, metallic, polyurethane, polyester, rubber, polyimide or any other natural or synthetic polymer that has been suitably treated to acquire the appropriate semiconducting properties. The semiconductor can be one that is n-type, one that is p-type, or one that is ambipolar.

Also included within the scope of a “thread” are other flexible substrates such as polyimide, paper, polyethylene terephthalate, parylene, polydimethylsiloxane, and other substances that be screen-printed, ink-jet printed, gravure printed, or laser patterned.

Suitable methods for making such threads are described in WO2017/023727, published on Feb. 9, 2017, the contents of which are incorporated herein by reference.

The portions of the first and second threads 12, 14 that are intended for electrical connection to instrumentation or power sources are made of any metallic or conducting material, such as gold, silver, carbon, copper, zinc, aluminum, platinum, or palladium, with or without a substrate or conductive polymer, PEDOT, polyaniline, polypyrrole, polyphenylene sulfide, poly(acetylene)s, polyphenylene vinylene. The carbon can be any carbon allotropes, such as graphite or graphene, or can be in the form of a carbon nanotube.

Examples of dielectric 18 include ion gel, ionic liquid, deep eutectic solvent mixtures, gels of ionic liquids, deep eutectic solvent mixtures, oxides, silicon dioxide, hafnium oxide, polymers, and electrolytes.

In some embodiments, the three-terminal sensor 10 is uncoated. In others, the three-terminal sensor 10 is coated with a suitable protective dielectric or insulating layer to protect the three-terminal sensor 10 from humidity or fouling. Examples of suitable materials include parylene, polydimethylsiloxane, polystyrene, and oxides.

1. A manufacture comprising a sensor for sensing a characteristic of a measurement region, said sensor comprising a first thread, a second thread, a dielectric, and a sensing electrode, wherein said second thread comprises a semiconductor, wherein said sensing electrode is configured to engage in an interaction with said measurement region, said interaction causing a change in an electrical characteristic of said sensing electrode, wherein said dielectric capacitively couples said first and second threads at a coupling region, and wherein said sensing electrode is in electrical communication with one of said first and second threads.

2. The manufacture of claim 1, wherein said sensing electrode is integral with one of said first and second threads.

3. The manufacture of claim 1, wherein said sensor electrode is integral with said first thread and wherein, in operation, an electric field resulting from interaction of said sensor electrode with said measurement region modulates a current on said second thread.

4. The manufacture of claim 1, wherein said sensor electrode is integral with said first thread, further comprising a third thread that is capacitively coupled with said second thread, wherein, in operation, an interaction of said sensor electrode with said measurement region causes a perturbation of an electric field caused by a potential applied between said first and third threads and wherein said perturbation modulates a current on said second thread.

5. The manufacture of claim 1, wherein said sensor electrode is disposed along a current path that includes said second thread and wherein, in operation, a change in an electrical property of said sensor electrode as a result of having interacted with said measurement region modulates a current along said current path.

6. The manufacture of claim 1, further comprising a loop that connects to said first thread, wherein said loop encircles said second thread in said coupling region.

7. The manufacture of claim 1, further comprising first and second wires knotted around said second thread, wherein said first and second wires are configured to connect to measurement instrumentation for sensing current in said second thread.

8. The manufacture of claim 1, further comprising third and fourth threads connected to said second thread, wherein said third and fourth threads are configured to connect to measurement instrumentation for sensing current in said second thread.

9. The manufacture of claim 1, further comprising first and second conducting paths connected to said second thread, wherein said sensor electrode is in electrical communication with said second thread through first conducting path and wherein, in operation, said second path connects to measurement instrumentation.

10. The manufacture of claim 1, further comprising a sensor fabric comprising a plurality of sensors, among which is said sensor, wherein said sensors in said sensor fabric comprise different kinds of sensors that are multiplexed such that an output of said sensor fabric corresponds to an output of a selected one of said sensors that comprise said sensor fabric.

11. The manufacture of claim 1, further comprising a sensor fabric comprising a plurality of sensors, among which is said sensor, wherein, for each sensor in said plurality, there exists a distance between a coupling region of said sensor and a sensor electrode for said sensor and wherein said distances differ among said sensors that comprise said sensor fabric.

12. The manufacture of claim 1, further comprising a sensor fabric comprising a plurality of sensors, each of which comprises a first thread, a second thread that defines a semiconducting portion of a current path, a dielectric that capacitively couples said first and second threads, and a sensing electrode that is along said current path, wherein said sensor is among said plurality of sensors that comprise said sensor fabric, wherein each of said sensing electrodes in said sensor fabric is configured to interact with said measurement region, said interaction causing a change in an electrical characteristic of said sensing electrode, wherein each of said conducting paths has a first end and a second end, wherein said conducting paths are shorted together at said second ends thereof, and wherein signals on each of said first threads cause all but one of said conducting paths to be in a non-conducting state, whereby an output of said sensor fabric represents a measurement made by a selected one of said sensing electrodes.

13. The manufacture of claim 1, further comprising a selection module and a sensor fabric comprising a plurality of sensors, each of which comprises a first thread, a second thread that defines a semiconducting portion of a current path, a dielectric that capacitively couples said first and second threads, and a sensing electrode that is along said first thread wherein said sensor is among said plurality of sensors, wherein each of said sensing electrodes is configured to interact with said measurement region, said interaction causing a change in an electrical characteristic of said sensing electrode, wherein each of said conducting paths has a first end and a second end, wherein said conducting paths are shorted together at said second ends thereof, wherein said selection module comprises transistors, each of which connects to a conducting path of a selected one of said sensors, and wherein signals on each of said transistors in said selection module cause all but one of said conducting paths to be in a non-conducting state, whereby an output of said sensor fabric represents a measurement made by a selected one of said sensing electrodes.

14. The manufacture of claim 1, further comprising a sensor fabric comprising a plurality of sensors, among which is said sensor, wherein among said sensors in said plurality of sensors are sensors that are members of a set of sensors that have been interconnected to form a logic gate and wherein each of said sensors that are members of said set comprises first and second threads capacitively coupled by said dielectric, said second thread defining a semiconducting current path.

15. The manufacture of claim 1, further comprising a sensor fabric comprising a plurality of sensors, among which is said sensor, wherein each of said sensors comprises capacitively coupled first and second threads and a sensor electrode along said second thread, said second thread defining a semiconducting current path, wherein among said sensors in said fabric are sensors that are members of a set of sensors that have been interconnected to carry out analog computation, wherein said analog computation comprises carrying out an operation on operands, and wherein said operands are defined by electrical characteristics of sensor electrodes from different sensors in said set of sensors.

16. The manufacture of claim 1, wherein said sensor electrode is configured such that said change in said electrical characteristic results from a change in temperature in sensed by said sensor electrode as a result of interaction with said measurement region.

17. The manufacture of claim 1, wherein said sensor electrode is configured such that said change in said electrical characteristic results from mechanical force experienced by said sensor electrode as a result of interaction with said measurement region.

18. The manufacture of claim 1, wherein said sensor electrode is configured such that said change in said electrical characteristic results from interaction of said sensor electrode with a chemical species in said measurement region.
